(19) United States
(12) Patent Application Publication
(10) Pub. No.: US 2009/0118586 A1
(43) Pub. Date: May 7, 2009

(54) Surgery accessory and method of use

(76) Inventor: Glenn A. Griffin, Corpus Christi, TX (US)

Correspondence Address: G. TURNER MOLLER, JR., 711 NORTH CARANCAHUA, SUITE 720, CORPUS CHRISTI, TX, 78475, US

(21) Appl. No.: 12/229,329

(22) Filed: Aug. 22, 2008

Related U.S. Application Data
(60) Provisional application No. 60/996,124, filed on Nov. 1, 2007.

(51) Int. Cl. A61B 17/34; B08B 9/04; A61M 31/00

(52) U.S. Cl. 600/204; 134/8; 604/26; 15/104.05

(57) ABSTRACT

In laparoscopic or thorascopic surgery, a trocar is used to provide an passage into the body cavity of a patient for injecting an inert gas to inflate it. A laparoscope or thorascope is passed through a sealing membrane of the trocar so the patient may be observed without releasing a large quantity of the inert gas. If the scope lens is clouded with body liquids or tissue, the scope is removed from the trocar and its lens cleaned. The trocar cannula is cleaned by running a swab through the sealing membrane into the cannula and removing body liquids, tissue and/or check valve lubricant from the inside of the cannula. The swab includes radiopaque markers on the sorbent end, measuring marks on the swab handle and an enlargement on the swab handle. The enlargement is positioned so it may be grasped and the swab pushed into the cannula a distance which is insufficient to push the sorbent end out of the end of the cannula.

This application is based on Provisional Application Ser. No. 60/996,124, filed Nov. 1, 2007 on which priority is claimed.

This invention relates to an accessory for assisting in the performing of laparoscopic or thorascopic surgery and a method of its use and more particularly to a method and apparatus for cleaning an laparoscopic or thorascopic trocar.

### BACKGROUND OF THE INVENTION

One of the great advances in surgery in the recent past has been the development of remote viewing surgery where the surgeon looks through or uses some type remote viewing instrument to see the site of the procedure. Laparoscopic, thorascopic, arthroscopic or endoscopic surgery are species of remote viewing surgery and involve the introduction of a viewing scope into the interior of a patient where a surgical procedure is to be performed. In the case of endoscopic surgery, an endoscope is inserted through a natural body opening. In the case of arthroscopic surgery, an incision is made near the joint where the procedure is to be done and a trocar inserted through the incision through which a viewing scope is inserted. In the case of all versions of remote viewing surgery except endoscopic surgery, a trocar is inserted through an incision in the patient's skin and a viewing scope and/or other instruments are passed through the trocar.

In the case of thorascopic surgery, a series of incisions are made in the chest wall to insert a variety of implements into the chest cavity. The only major difference between a thorascopic trocar and a laparoscopic trocar is the length of the cannula, i.e. the thorascopic trocars have cannulas which are several inches shorter than laparoscopic trocars. In the case of laparoscopic surgery, several small incisions are made in the patient's abdominal wall to insert a variety of implements into the patient's abdomen. A series of trocars are inserted into the abdomen through the incisions to provide one or more pathways for a laparoscope and/or other surgical instruments. After removing a piercing implement or obturator from the trocar, the abdomen or chest is distended by injecting an inert gas, typically carbon dioxide, through the cannula of the trocar thereby providing some room to perform a surgical procedure. Each trocar includes a sealing membrane having an opening therein for passing the laparoscope or other surgical implements into the patient's abdomen while largely preventing the escape of the inert gas from the patient's abdomen or chest cavity. In addition, some trocar embodiments include a check valve of some type to prevent the escape of the inert gas from the patient's abdomen when the laparoscope or other surgical implement is removed. Some trocar models include a flapper type check valve while others include a slit diaphragm but both have the function of reducing or preventing gas escape from the patient's abdomen or chest cavity when the laparoscope or surgical implement is removed. The number and placement of the incisions and the particular type of laparoscope and surgical instruments depends, of course, on the type and extent of surgery to be performed.

In the case of thorascopic surgery, one or more small incisions are made in the patient's chest cavity. One or more trocars are inserted into the chest cavity so a thorascope and/or other surgical instruments may be inserted into the patient. So far as is relevant for purposes of this invention, the laparoscopic and thorascopic procedures are essentially the same. As used herein, the word torso is used as a generic term to include the abdomen and the chest cavity.

Periodically, the lens on the forward end of the laparoscope may be obscured or partly obscured by body liquids, tissue or lubricant on the check valve. In current practice, the surgeon withdraws the laparoscope from the cannula, cleans the lens and reinserts the laparoscope through the cannula of the trocar. Occasionally, the interior wall of the cannula collects body liquids, tissue or check valve lubricant, meaning that the lens may become clouded or obscured when reinserted through the cannula. Currently, the cannula is attempted to be cleaned with surgical gauze.

It has been proposed in the prior art to provide for in situ cleaning of laparoscope lenses or to provide lens washing nozzles on laparoscopes as shown and discussed in U.S. Pat. No. 5,392,766. Although these approaches may theoretically be workable, they have not been accepted, for whatever reasons, by general surgeons in normal every day surgery.

It has been proposed in the prior art to clean the inside of arthroscopes used in non-abdominal surgery with a swab as shown in U.S. Pat. Nos. 5,029,573; 5,318,582 and 5,356,419.

Other disclosures of some interest relative to this invention are found in U.S. Pat. Nos. 3,133,538; 3,205,518; 3,376,867; 4,626,251; 5,928,176; 6,045,623 and D401,326 along with Printed Patent Application 2005/0267,421.

### SUMMARY OF THE INVENTION

In this invention, the inside of a cannula of a laparoscopic or thorascopic trocar is cleaned with a swab in the middle of surgery to remove body liquids, tissue or check valve lubricant from the inside of the cannula. Typically, the laparoscope lens becomes clouded and the laparoscope is removed from the cannula and cleaned. Before the laparoscope is reinserted through the cannula, the swab of this invention is run through the cannula to remove any body liquids, tissue or check valve lubricant thereby allowing the lens to generate clear pictures to the monitor being watched by the surgeon.

The swab is of unusual design and includes a handle of sufficient size to seal against the sealing membrane opening of the cannula to thereby prevent the escape of gas from the patient's abdomen or chest cavity when the swab is being used to clean the inside of the cannula. The swab handle includes depth markings to the end of the sorbent patch on the handle end and/or a shoulder or enlargement at a location where the swab is fully inserted into the cannula. In effect, the enlargement acts as a depth gauge to minimize the potential of dislodging the sorbent patch from the handle and leaving the sorbent patch inside the patient. Radiopaque elements on the sorbent swab end allow the swab end to be located by x-ray or fluoroscope.

It is an object of this invention to provide an improved method and apparatus for cleaning a laparoscopic or thorascopic cannula during abdominal surgery or thoracic surgery.

A further object of this invention is to provide an improved swab for cleaning a laparoscopic or thorascopic cannula during surgery.

A more specific object of this invention is to provide an improved swab having a handle providing an enlargement to minimize the possibility of the swab being inserted past the end of the cannula.

These and other objects and advantages of this invention will become more apparent as this description proceeds, reference being made to the accompanying drawings and appended claims.

### BRIEF DESCRIPTION OF THE DRAWINGS

FIG. 1 is an exploded isometric view of a conventional arthroscopic trocar;

FIG. 2 is an exploded cross-sectional isometric view of a conventional arthroscopic trocar;

FIG. 3 is an exploded isometric view of a conventional laparoscopic trocar;

FIG. 4 is an exploded cross-sectional isometric view of a conventional laparoscopic or thorascopic trocar;

FIG. 5 is an enlarged exploded view of the trocar of FIG. 3, many of the long components being cut off for purposes of illustration;

FIG. 6 is a pictorial view of one embodiment of a swab of this invention;

FIG. 7 is a pictorial view of another embodiment of a swab of this invention.

### DETAILED DESCRIPTION OF THE INVENTION

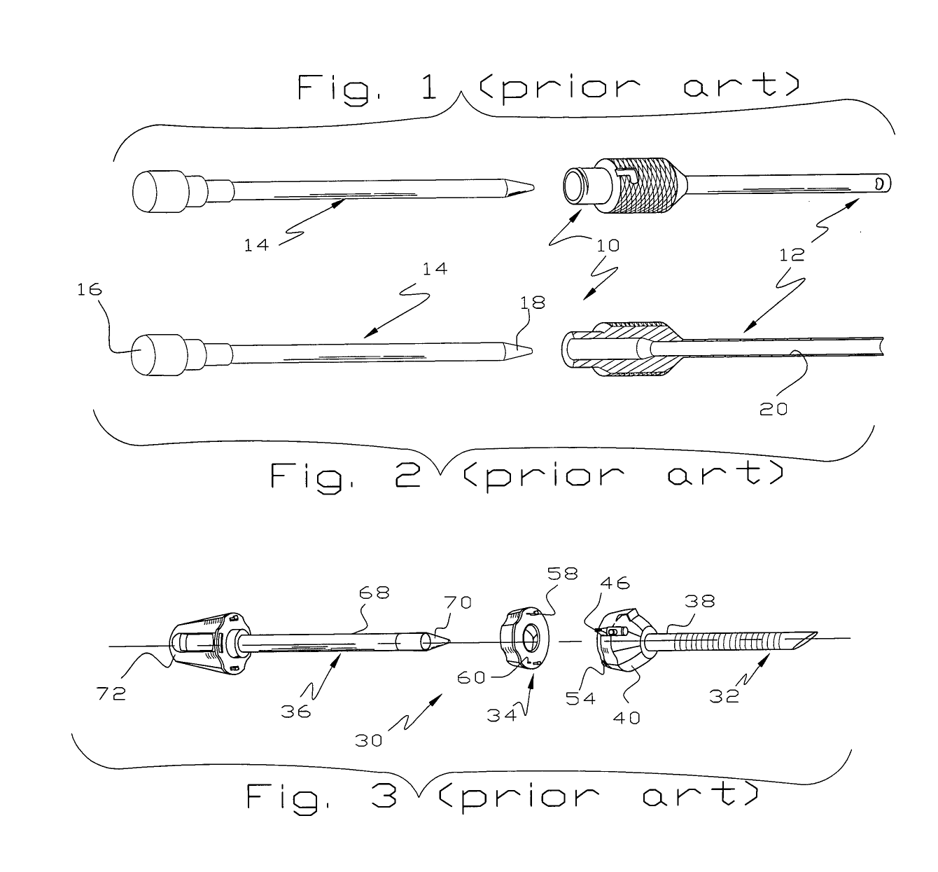

Referring to FIGS. 1-2, a conventional arthroscopic trocar 10 comprises a cannula 12 into which is inserted a piercing implement or obturator 14. In use, an incision is made in the patient, as on the back of the hand as shown in U.S. Pat. Nos. 5,029,573; 5,318,582 and 5,356,419 and the trocar 10 inserted into the incisions. The trocar 10 is advanced into the patient's body by pushing on an end 16 of the piercing element so the point 18 burrows its way through the patient's flesh to reach the desired location where a surgical procedure is to be conducted. The obturator 14 is then removed, leaving the cannula 12 in place. A series of the trocars 10 may be placed in the patient, depending on the type and extent of surgery to be performed. The arthroscopic trocar 10 will be seen to be quite simple and the cannula 12 is of small internal diameter. Although there may be some variation in the size of the passage 20 through the cannula 12, they are very small and usually are in the 4 millimeter range.

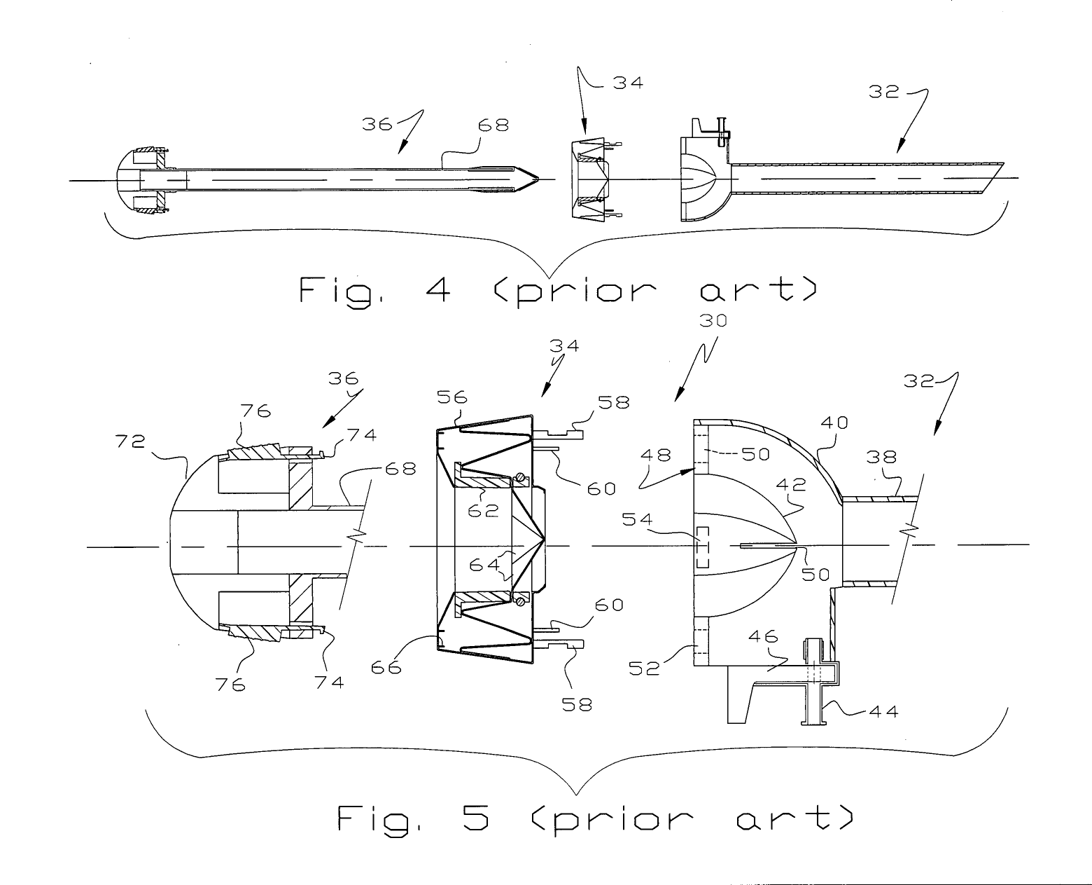

Referring to FIGS. 3-5, a conventional laparoscopic trocar 30 comprises, as major components, a cannula 32, a sealing section 34 and a piercing element or obturator 36. In some embodiments, the cannula 32 may comprise an elongate exteriorly ribbed conduit 38 having a bulbous end 40 housing a slit diaphragm type check valve 42, a port 44 having a valve 46 for delivering and withdrawing inert gas to and from the patient's abdomen and a latching mechanism 48 for securing the sealing section 34 to the cannula 32. The slit diaphragm 42 conveniently includes a single slot 50 through which the laparoscope (not shown) extends. When the laparoscope is removed, the size and shape of the diaphragm 42 closes to prevent flow of the inert gas out of the patient.

The latching mechanism 48 may be of any suitable type and conveniently includes openings 52 for receiving prongs on the sealing section 34. An actuator 54 rotates a spring biased ring (not shown) to release the prongs in order to separate the sealing section 34 and the cannula 32.

In some embodiments, the sealing section 34 may comprise a housing 56 having finger prongs 58 received in the openings 52 to secure the sealing section 34 and the cannula 32 together. One or more guide pins 60 may be provided on the sealing section 34 to be received in guide openings (not shown) to align the sealing section 34 and the cannula 32.

In some embodiments, the sealing section 34 also comprises a floating ring 62 carrying a series of sealing elements 64 providing a central opening through which the piercing element 36 extends. The sealing elements 64 comprise a series of rubber flaps that act much like the iris of an eye to spread apart when the obturator 36 passes through them thereby sealing on the periphery of the obturator 36. The sealing section 34 also comprises openings 66 for latching onto the obturator 36.

The obturator 36 comprises an elongate shaft 68 having a pointed end 70 which is sharp enough to penetrate tissue below the skin of the patient. The obturator 36 includes a dome shaped end 72 which can be easily grasped by the surgeon and pushed through the abdominal wall of the patient. One or more latches 74 align with and enter the openings 66 of the sealing section 34 for securing the obturator 36 to the sealing section 34. The latches 74 may be retracted by pressing on buttons 76. Laparoscopic trocars typically have cannulas of considerably larger internal diameter in order to pass cameras, surgical implements and the like which are necessitated by laparoscopic surgery. While the internal diameters of laparoscopic cannulas may vary from 5-15 millimeters, the vast majority of laparoscopic cannulas have internal diameters of 8-12 millimeters. Those skilled in the art will recognize the trocar 30 to be a conventional trocar made by Ethicon Endo-Surgery of Somerville, N.J., and known as an Endopath XCEL, size 12, which means it is twelve millimeters outside diameter.

Use of the laparoscopic trocar 30 should now be apparent. After the surgeon makes the necessary incision in the patient's skin, the pointed end 70 of the assembled trocar 30 is inserted through the incision and pushed to pass through the patient's abdominal wall. When the trocar 30 has been inserted to the appropriate location, the obturator 36 is removed by pressing on the buttons 76 and unlatching the obturator 36 from the sealing section 34. The surgeon then inflates the patient's abdomen by connecting a source of pressurized inert gas to the port 44 and opening the valve 46. When the patient's abdomen has been distended sufficiently, the surgeon closes the valve 46 thereby disconnecting the source of pressurized inert gas. Depending on the nature of the surgical procedure being conducted, the surgeon may make other incisions in the patient's abdomen and position additional trocars at strategic locations to assist in the conduct of the surgical procedure.

Sooner or later, the surgeon passes a laparoscope through one of the trocars and views the operation site on a monitor in the operating room. Surgical steps are taken by inserting implements through others of the trocars and the surgical procedure is conducted, depending on the nature and extent of the procedure. Occasionally, the lens on the laparoscope will become clouded by body liquids, tissue and/or check valve lubricant. The surgeon may remove the laparoscope from the trocar 30 and clean the lens with any available sorbent material. Occasionally, detritus such as body liquids, tissue and/or check valve lubricant accumulates on the inside of the cannula 38 so reinsertion of the laparoscope through the cannula 38 may again cause the lens to be fouled so the picture on the monitor being watched by the surgeon is not clear. Those skilled in the art will recognize the laparoscopic procedure, as described above, to be conventional.

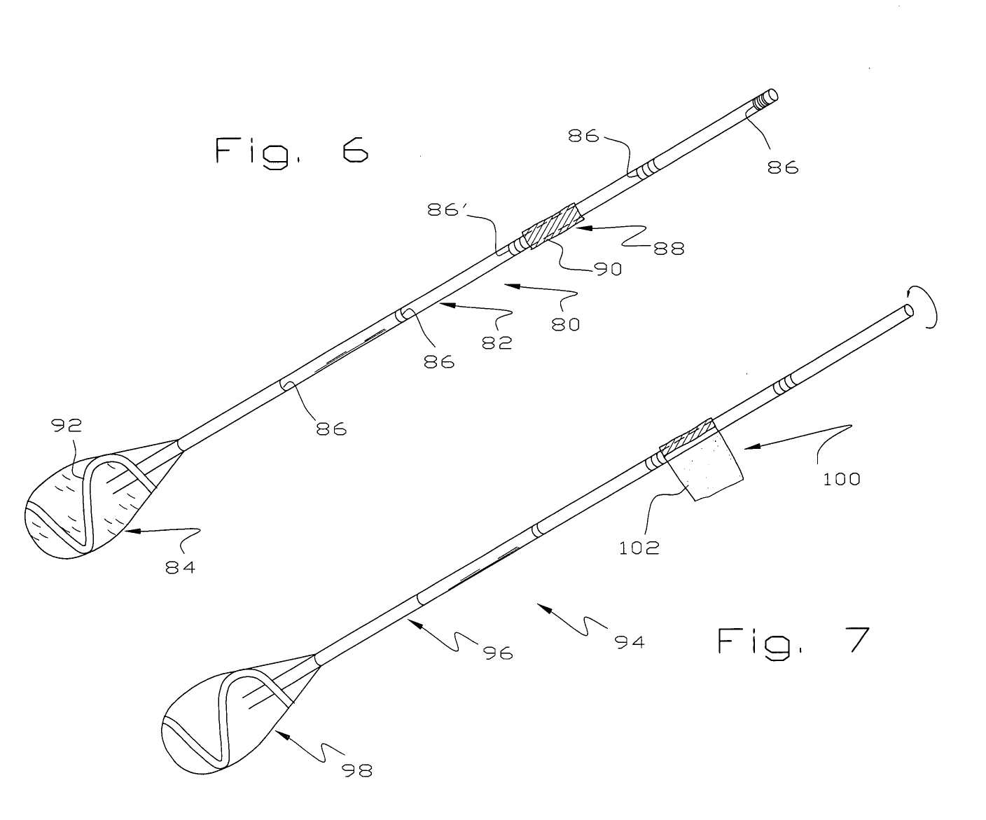

In the event the cannula 38 has accumulated body liquids, tissue and/or check valve lubricant which may cloud the laparoscope lens, a swab 80 is provided that is designed to clean the inside of the cannula 38 as shown in FIG. 6. The swab 80 comprises a handle 82 and an sorbent end 84. The handle 82 is of substantial diameter, both to support the weight of the sorbent end 84 and to seal against the sealing elements 64 to inhibit the loss of the inert gas from the patient's abdomen. Typically, the handle 82 has an outer diameter of 3-4 millimeters although it will be understood that any diameter which is strong enough to be relatively inflexible, support the load of the sorbent end 84 and seal against the sealing elements 64 will be operative in this invention.

The handle 82 preferably includes a variety of unusual features. In some embodiments, a series of distance measuring marks or indicia 86, 86′ can be provided to indicate the distance from the far or distal end of the sorbent end 84 to the mark 86, 86′. Preferably, the marks 86 are located at distances from the sorbent end 84 corresponding to the lengths of cannula that are compatible with the particular swab 80. This may be used to prevent the surgeon from inserting the swab 80 so far into the cannula 38 that the sorbent end 84 passes out of the end of the cannula 38. This is not desirable because it creates the possibility that the sorbent end 84 may become detached from the handle 82 requiring it to be retrieved from inside the patient. For example, the Ethicon Endo-Surgery Model Endopath XCEL, size 12 discussed above, the distance from the proximate end of the sealing section 34 to the distal end of the cannula 38 is approximately nineteen centimeters. For use with this particular trocar, the mark 86′ is preferably slightly less than nineteen centimeters from the end of the sorbent end 84.

In some embodiments, the handle 82 can include a tactile distance device or indicator 88 such as a shoulder or enlargement 90 to indicate the distance from the sorbent end 84 to the mark 86′. In some embodiments, the enlargement 90 is permanently fixed to the handle 82 at the time of manufacture. The enlargement 90 may be used to prevent the surgeon from inserting the swab 80 so far into the cannula 38 that the sorbent end 84 passes out of the end of the cannula 38. For use with the Ethicon Endo-Surgery Model Endopath XCEL described above, the mark 86′ is preferably about nineteen centimeters from the end of the sorbent end 84. In use, the surgeon can grasp the enlargement 90 and push the swab 80 through the cannula 38 and confidently realize that the sorbent end 84 will not pass out of the cannula 38.

The sorbent end 84 may be of any suitable type to remove debris or detritus from the inside of the cannula 38 and can be either adsorbent or absorbent materials. Materials, such as natural or polymer fibers, foam or the like are preferable. There is some tendency for fibers from the sorbent end 84 to be pulled loose when passed through the sealing elements 64 and/or the check valve 42. Accordingly, some care in the manufacture of the sorbent end 84 from fibers is preferably done to minimize pulling fibers from the sorbent end. In some embodiments, one or more radiopaque threads 92 are incorporated into the sorbent end 84 so the location of the sorbent end 84 may be determined by x-ray or fluoroscope in the unlikely event it becomes detached from the handle and is inside the patient.

Referring to FIG. 7, another embodiment of a swab 94 is illustrated. The swab 94 includes a handle 96 and sorbent end 98 having similar characteristics to the handle 82 and sorbent end 84 although the end 98 is illustrated as being foam rather than fiber. In the swab 94, a tactile distance indicator 100 can be attached to the handle 96 in the operating room by one of the operating room personnel, such as a surgical technician, nurse or surgeon. To determine the location of the indicator 100, the swab 94 is inserted into the cannula 38 until the sorbent end 98 begins to exit the end of the cannula 38 and the indicator 100 is attached to the handle 96. To this end, the indicator 100 can be an adhesive backed tape 102 and, depending on the thickness of the tape, can be wound multiple times around the handle 96 to provide the desired thickness.

By applying the indicator 100 at or near the time of surgery, one can significantly reduce the number of models of the swab of this invention in order to accommodate cannulas of significantly different length and diameter. For example, the sorbent end 98 can be made the same size to clean cannulas 38 having internal diameters between 5-15 millimeters and, by applying the indicator 100 in the operating room or near the time of surgery, only one size swab is needed. If the indicator 100 were permanently attached to the handle 96 at the time of manufacture, every model trocar would have to have a swab made specifically for it.

It is estimated there are about 35,000 surgeons in the United States who routinely do remote viewing surgery of all types. It is estimated that these surgeons currently do an average of 100 remote viewing surgeries per year for a total of about 3,500,000. Although remote viewing surgery has been increasing in popularity, it would not be surprising to learn that 30,000,000 remote viewing surgeries have been conducted in the United States in the last ten years.

It will accordingly be seen that the same cleaning operations conducted during laparoscopic procedures conducted in the abdomen of the patient may be conducted during thorascopic procedures conducted in the chest cavity of the patient.

The method of cleaning a laparoscope or thorascope as described has not been previously known. It is the spirit and scope of this invention to provide a new method and apparatus for cleaning laparoscopic and/or thorascopic trocars.

Although this invention has been disclosed and described in its preferred forms with a certain degree of particularity, it is understood that the present disclosure of the preferred forms is only by way of example and that numerous changes in the details of operation and in the combination and arrangement of parts may be resorted to without departing from the spirit and scope of the invention as hereinafter claimed.

I claim:

1. A method of performing remote viewing surgery, comprising
creating an incision in a patient's torso;
passing through the incision a trocar having a passage therethrough and a sealing membrane in the passage having an opening therein for sealing against elongate objects moved through the membrane and reducing the rate of gas escape from the patient's torso;
injecting an inert gas through the passage and distending the patient's torso;
passing a laparoscope through the passage and sealing its exterior with the membrane;
removing the laparoscope from the passage; and
passing a swab through the membrane opening and through the passage and cleaning detritus from the inside of the passage.

2. The method of claim 1 wherein the swab includes a sorbent end and a handle of smaller thickness than the sorbent end and wherein the step of passing the swab through the membrane comprises expanding the membrane opening with the sorbent swab end.

3. The method of claim 2 wherein the swab comprises a handle and the step of passing the swab through the membrane comprises sealably engaging the membrane with the swab handle.

4. The method of claim 1 wherein the swab includes a sorbent swab end comprising cotton strands and the step of cleaning detritus from the inside of the passage comprises collecting the detritus on the cotton strands.

5. The method of claim 1 wherein the swab includes a sorbent swab end comprises a foam and the step of cleaning detritus from the inside of the passage comprises collecting the detritus on the foam.

6. The method of claim 1 wherein the swab includes a sorbent end and a handle having markings thereon indicating a distance to the sorbent end and wherein the step of passing the swab through the passage comprises using the markings to extend the handle into the trocar a distance less than a distance from a distal end of the trocar passage and a proximal end of the trocar passage.

7. The method of claim 1 wherein the swab includes a sorbent end and a handle having an enlargement thereon at a distance to the sorbent end less than a distance from a distal end of the trocar passage and a proximal end of the trocar passage and wherein the step of passing the swab through the passage comprises using the enlargement to control the distance the handle is pushed into the trocar passage.

8. The method of claim 7 wherein the using step comprises grasping the enlargement and pushing the swab into the trocar passage until the enlargement reaches a proximal end of the trocar passage.

9. The method of claim 7 further comprising wrapping a elongate member around the handle to provide the enlargement.

10. The method of claim 9 wherein the laparoscopic surgery is conducted in an operating room and the wrapping step is conducted in the operating room.

11. The method of claim 10 wherein the laparoscopic surgery is conducting by an operating room team and the wrapping step is conducted by a member of the operating room team.

12. The method of claim 1 wherein the swab includes a sorbent end having a radiopaque mark thereon.

13. The method of claim 1 wherein the trocar comprises a check valve in the passage between the sealing membrane and a distal end of the passage and wherein the step of passing the swab through the passage toward a distal end of the passage comprises pushing the check valve from a closed position to an open position.

14. The method of claim 1 wherein the incision is made in the patient's abdomen and the step of injecting an inert gas through the passage comprises distending the patient's abdomen.

15. The method of claim 1 wherein the incision is made in the patient's chest and the step of injecting an inert gas through the passage comprises distending the patient's chest.

16. A swab for cleaning the inside of a trocar passage during remote viewing surgery, comprising a sorbent end, a handle and an enlargement on the handle at a location intermediate the ends thereof for aiding in controlling the distance the swab is pushed into a trocar passage.

17. The swab of claim 16 wherein the handle provides a series of distance marks indicating the distance from a distal end of the sorbent end to the mark.
